(19) United States
(12) Patent Application Publication
(10) Pub. No.: US 2006/0014684 A1
(43) Pub. Date: Jan. 19, 2006

(54) Novel uses of EGF

(75) Inventors: Andre G. Buret, Calgary (CA); D. Grant Gall, Calgary (CA); Merle E. Olson, Calgary (CA); James A. Hardin, Calgary (CA)

Correspondence Address: Eileen McMahon;Torys LLP, 79 Wellington Street West, Box 270 TD Centre, Toronto, ON, M5K 1N2, CA

(73) Assignee: University Technologies International Inc., Calgary (CA)

(21) Appl. No.: 11/009,490

(22) Filed: Dec. 10, 2004

Related U.S. Application Data
(63) Continuation of application No. 10/280,130, filed on Oct. 25, 2002.
Application No. 10/280,130 is a continuation-in-part of application No. 09/518,135, filed on Mar. 3, 2000, now Pat. No. 6,656,907.

(51) Int. Cl. A61K 38/18

(52) U.S. Cl. 514/12

(57) ABSTRACT

This invention relates to treating or preventing pathogenic infections with an epidermal growth factor (EGF). EGF is capable of inhibiting pathogenic colonization of pathogens in a variety of tissue or cell types. Since pathogenic colonization is essential for pathogenic infection, EGF can be used as an effective preventive and therapeutic agent for pathogenic infections, particularly in the urogenital tract. A method of increasing weight gain in an animal by administering epidermal growth factor is also described.

### RELATED APPLICATION

This application is a continuation of Ser. No. 10/280,130, filed 25 Oct. 2002, which is a continuation-in-part of U.S. patent application Ser. No. 09/518,135, filed 3 Mar. 2000, now U.S. Pat. No. 6,656,907, which is a continuation of Ser. No. 08/945,760, filed as international application no. PCT/CA96/00291 on 9 May 1996, abandoned, which is a continuation-in-part of U.S. application Ser. No. 08/438,901, filed 10 May 1995, now U.S. Pat. No. 5,753,622.

### FIELD OF THE INVENTION

This invention relates to treating or preventing pathogenic infections with an epidermal growth factor. This invention also relates to a method of promoting weight gain and preventing gastrointestinal colonization by pathogens by oral administration of epidermal growth factor.

### REFERENCES

U.S. Pat. No. 5,547,935.
U.S. Pat. No. 5,753,622.
U.S. Pat. No. 6,191,106.
U.S. Patent Application Publication No. 20020098178A1.
Buret, A., M. E. Olson, D. G. Gall, and J. Hardin, “Effects of orally administered epidermal growth factor on enteropathogenic Escherichia coli infection in rabbits”, Infect. Immun. 66: 4917-4923 (1998).
Carpenter et al., “Epidermal growth factor”, Ann. Rev. Biochem. 48: 193-216 (1979).
Domingue, Sr., G. J. et al., “Prostatitis”, Clinical Microbiology Rev. 11(4): 604-613 (1998).
Elliott, S. N. et al., “Bacteria rapidly colonize and modulate healing of gastric ulcers in rats”, Am. J. Physiol. Gastrointest. Liver Physiol. 275: G425-G432 (1998).
Goodlad, R. A. et al. (1991) “Effects of Urogastrone-Epidermal Growth Factor on Intestinal Brush Border Enzymes and Mitotic Activity”, Gut 32(9): 994-998.
Gregory, H. “In vivo aspects of urogastrone-epidermal growth factor”, J. Cell Sci. 3: 11-17 (1985).
Konturek et al., “Role of growth factors in gastroduodenal protection and healing of peptic ulcers”, Gastroenterol. Clin. North Am. 19: 41-65 (1990).
O'Loughlin, E. V. et al. (1985) “Effect of Epidermal Growth Factor on Ontogeny of the Gastrointestinal Tract”, Am. J. Physiol. 249:G674-G678.
O'Loughlin, E. V. et al. (1994) “Structural and Functional Adaptation Following JeJunal Resection in Rabbits: Effect of Epidermal Growth Factor”, Gastroenterology 107:87-93.
Okabe, S. and C. J. Pfeiffer, “Chronicity of acetic acid ulcer in the rat stomach”, Am. J. Dig. Dis. 17: 619-629 (1972).
Rosamilia, A. et al., “Pathophysiology of interstitial cystitis”, Current Opin. Obstetrics and Genecology 12: 405-410 (2000).
Walker-Smith, J. A. et al. “Intravenous Epidermal Growth Factor/Urogastrone Increases Small Intestinal Cell Proliferation in Congenital Microvillous Atrophy”, Lancet 2(8466):1239-1240 (1985).

All the publications, patents and patent applications cited above or elsewhere in this application are herein incorporated by reference in their entirety to the same extent as if the disclosure of each individual publication, patent application or patent was specifically and individually indicated to be incorporated by reference in its entirety.

### BACKGROUND OF THE INVENTION

Mucosal surfaces are the wet, inner linings of internal ducts of animals which are connected with the outside environment, including for example the entire digestive tract (from the oro-nasal cavity to the anus), the respiratory tract, the uro-genital tract, the ocular surface, the mammary glands and the prostate. Mucosal surfaces are covered by epithelial cells, most often simple column epithelia or stratefied epithelia, and often secrete mucus. Because of its frequent contact with the outside environment, the mucosal surface is particularly susceptible to pathogenic infection.

Pathogenic infections begin with pathogenic adhesion and colonization, of which the mechanism is not clear. However, microbial pathogens typically require binding to the host cell surface in order to develop an efficient infection. Following adhesion and colonization, microorganisms multiply on the colonized surface and/or invade the host cell. While not necessarily sufficient to cause a disease, this interaction between the pathogen and the host is a determining factor in microbial pathogenicity. Therefore, inhibiting pathogenic colonization would be an efficient way of preventing and/or treating pathogenic infections.

Currently, antibiotics are the most widely used anti-infectious agents against pathogens. Antibiotics are typically efficient inhibitors of bacterial growth or replication which can quickly alleviate symptoms of diseases caused by bacterial infection. However, due to the overuse of antibiotics, many bacteria have developed resistance to antibiotics, and the number of antibiotics that can be used has dramatically decreased. In addition, antibiotics are effective against bacteria, but it is still difficult to treat infections caused by other pathogens such as viruses. Therefore, there remains a need for anti-infectious agents against pathogens.

A number of intestinal growth factors accelerate epithelial maturation and renewal. One of these, epidermal growth factor (EGF) is naturally present in salivary and intestinal secretions and other body fluids, and is produced in large quantities in colostrum and milk. EGF promotes a) the proliferation and differentiation of intestinal cells during early life, b) the functional maturation of the pre-weaning intestine, and c) epithelial proliferation in the adult gut (Weaver, et al. Gastroenterology 1990;98:828-837; O'Loughlin, et al. Am J Physiol 1985;249:G674-G678; Goodlad, et al. Gut 1991;994-998; Walker-Smith, et al. Lancet 1985;ii:1239-1240; Hardin, et al. Am J Physiol 1993;264:G312-G318). Moreover, EGF acutely (within minutes) upregulates small intestinal absorption of electrolytes and nutrients, an effect which was shown to be related to a concurrent lengthening of the apical microvilli of enterocytes (O'Loughlin, et al. Gastroenterology 1994;107:87-93). Potential therapeutic benefits of EGF have been highlighted in studies where topical treatment with EGF promoted wound healing (Brown, et al. New Engl. J. Med. 1989;321(2):76-79) and, more recently, by the observation that administration of EGF enhances nutrient absorption in remnant intestine following massive resection (Pothier and Menard FEBS Lett. 1988;228(1) 113-117). Compared with the small intestine, more receptors for EGF are found in the colon (Brake, et al. Proc Natl Acad Sci USA 1984;81:4642-4646), where the heaviest bacterial load is observed during infection with microorganisms such as Esherichia coli. EGF upregulates function in the entire intestine, including the colon (Goodlad, et al. Gut 1991;994-998; Pothier and Menard, FEBS Lett. 1988;228(1) 113-117).

While EGF has been reported to have a variety of functions, its role in preventing intestinal colonization by pathogens or in accelerating weight gain have not been previously reported. These two newly discovered properties of EGF make it extremely useful as a therapeutic agent in young farm animals.

In addition to demonstrating that administration of EGF can prevent intestinal colonization by pathogens, the inventors have also shown that EGF can enhance weight gain in animals. The latter effect is unexpected as certain publications have indicated that EGF has no effect on weight gain (Bird, et al. J Nutr 1994;124:231-240; Opleta-Madsen, et al. Am J Physiol 1991;260:G807-G814). Other studies investigating the effects of EGF in pigs (James, et al. J Physiol 1987;393:569-582; Jaeger, et al. Am. J. Vet. Res. 1990;5(3):471-474) were unable to demonstrate an acceleration in growth rate, despite concurrent increases in the levels of intestinal disaccharidases.

As discussed above, EGF causes an increase in the intestinal absorption of nutrients. On the other hand, inhibition of the EGF signalling cascade reduces intestinal absorption of nutrients. However, the clinical benefits of inhibiting the EGF signalling cascade in the regulation of gastrointestinal nutrient absorption have never been assessed. It is predicted that antagonists of the EGF receptor or the EGF signalling cascade may be used as a gastrointestinal therapeutic agent where decreased intestinal absorption may be warranted for example in treating obesity, or to decrease intestinal uptake of toxic or adverse substances.

### SUMMARY OF THE INVENTION

The present invention relates to the use of epidermal growth factor (EGF) as a gastrointestinal therapeutic agent.

Epidermal growth factor (EGF) has been shown to inhibit pathogenic colonization in the gastrointestinal tract (U.S. Pat. No. 5,753,622). It is well documented that EGF is present in large amounts in, and has diversified biological activities on, the gastrointestinal tract. Therefore, the inhibitory effect of EGF on pathogenic colonization in this tract suggests that EGF may specifically recognize and interact with the epithelial cells in the gastrointestinal tract, thereby interfering with the interaction between pathogens and the epithelial cells. Surprisingly, we discovered that EGF can also inhibit pathogenic colonization in other tissue and organ types, including bladder and kidney. Our findings therefore indicate that EGF is an effective preventive or therapeutic agent against pathogenic infections in a wide variety of tissue and organ types, particularly the urogenital tract.

Accordingly, one aspect of the present invention provides a method of inhibiting or treating a pathogenic infection of the urogenital tract in an animal, comprising administering an effective amount of an epidermal growth factor (EGF) to the animal. In particular, the infection may be a bacterial, yeast, parasitic or viral infection.

In another aspect of the present invention, EGF may be used to treat or prevent pathogenic infections which are the etiological factors of a disease or condition, although the pathogen(s) may not have been identified, and/or the infections are subclinical in the disease or condition. In particular, the disease or condition is prostatitis or cystitis. The prostatitis may be acute bacterial prostatitis, chronic bacterial prostatitis, or chronic idiopathic prostatitis. Preferably, the prostatitis is bacterial prostatitis (acute or chronic). The cystitis may be bacterial cystitis or interstitial cystitis, and is preferably bacterial cystitis.

The epidermal growth factor can be administered by any method established in the art, preferably administered topically or systemically, and more preferably administered topically. The epidermal growth factor may be any polypeptide which has substantial amino acid sequence identity with the native EGF while possessing the EGF biological activity, and is preferably selected from the group consisting of the native EGF, EGF51 gln51, EGF-D, EGF-X₁₆, HB-EGF, TGF and the fusion proteins thereof.

The pathogenic infection may be subclinical or symptomatic. The infection may also be secondary to a disease or medical condition. In particular, the infection may occur subsequent to a wound. Any wound which is susceptible to pathogenic infections is contemplated in the present invention. The wound is preferably located in the skin or a mucosal surface. In particular, the wound is selected from the group consisting of burns, cuts, punctures, ulcers or tears.

In another aspect, the present invention provides a method of increasing weight gain in an animal.

In yet a further aspect, the present invention provides a method of decreasing intestinal absorption of nutrients which comprises administering an agent that inhibits the activity of EGF to said animal. Such a method may be useful in situations where decreased intestinal absorption is desired such as in treating obesity or in decreasing the intestinal absorption of toxins.

The EGF is preferably administered orally, for example in the feed of the animal. Further, lyophilized EGF added to drinking water has proven stable and therefore can be administered as such.

### DESCRIPTION OF THE DRAWING

FIG. 1 is a graph showing the effect of EGF on weight gain in rabbits.

### DETAILED DESCRIPTION OF THE INVENTION

This invention relates to treating or preventing pathogenic infections with an epidermal growth factor (EGF). EGF has been shown to inhibit pathogenic colonization in the gastrointestinal tract, and this phenomenon is consistent with its abundance and diversified biological activities in the gastrointestinal tract. Surprisingly, we discovered that EGF can also inhibit pathogenic colonization in other tissues or organs, including bladder and kidney. Our findings therefore indicate that EGF is an effective preventive or therapeutic agent against pathogenic infections in a wide variety of tissue and organ types.

EGF has been shown to prevent gastrointestinal colonization by pathogens and to promote weight gain in animals. Consequently, EGF is a very useful agent that can be used to increase production in the animal industry such as the beef, pig and poultry industry. In addition, EGF treatment may have clinical benefits in humans (i.e. during Crohn's disease, gastrointestinal infection, traveller's diarrhea, etc. Inhibitors of EGF may decrease nutrient absorption in the intestine and as such may be useful in treating obesity or in preventing absorption of toxins.

Prior to describing the invention in further detail, the terms used in this application are defined as follows unless otherwise indicated.

Definitions

“Inhibiting or treating” a pathogenic infection means reducing the extent of infection either after the onset of the infection or as a prophylactic treatment. The extent of infection may be determined by any established method in the art, for example by observing the symptoms associated with the infection or by culturing and calculating the number of pathogens present at the infection site. The extent of infection is reduced preferably by at least about 10%, more preferably by at least about 20%, yet more preferably by at least about 30% and most preferably by at least about 50%.

A “pathogen” is any microorganism capable of infecting an animal. Examples of pathogens include, but are not limited to bacteria, fungi (including yeast), viruses and protozoan parasites.

A “pathogenic infection” is an infection caused by a pathogen. An infection refers to a condition whereby the pathogen proliferates in the host animal and/or generates pathogenic products to result in the symptoms associated with the infection. Examples of such pathogenic products are the toxins made by bacteria.

A “mucosal surface” or “mucosa” is the lining of body cavities that are open to the exterior, such as the digestive tract, respiratory tract, or urogenital tract. In all cases, the mucosa is a wet, or moist, surface bathed by secretions or, in the case of the urinary mucosa, urine. All mucosae consist of an epithelial sheet directly underlain by a lamina propria, a layer of loose connective tissue just deep to the basement membrane. The cell compositions of mucosae vary. However, the majority of mucosae contain either stratified squamous or simple columnar epithelia. Although many mucosae secrete mucus, this is not a requirement. The mucosae of the digestive and respiratory tracts secrete large amounts of protective lubricating mucus, but those of the urinary tract do not. Other examples of mucosae can be found at, without being limited to, the ocular surface, mammary glands and prostate.

The “urogenital tract” means the urinary and genital organs and the associated structures, including kidneys, ureters, bladder, urethra, and genital structures of the male and female. In the female animals, the genital structures include the ovaries, fallopian tubes, uterus, cervix, and vagina. In the male animals, the genital structures include the testes, seminal vesicles, seminal ducts, prostate, and penis.

A “wound” is a bodily injury caused by physical means which resulted in disruption of the normal continuity of structures. Particularly included as wounds are burns, cuts, punctures, ulcers and tears of the skin or mucosal surfaces.

An “effective amount” is an amount sufficient to achieve its intended purpose. For example, an effective amount of EGF to inhibit or treat a particular E. Coli infection is an amount sufficient to reduce the symptoms or number of E. coli associated with the infection. The effective amount will vary with factors such as the route of administration, the form of EGF administered, the animal being treated, the nature of the pathogen and the infection. Therefore, the effective amount needs to be empirically or clinically determined according to established methods in the art.

An “epidermal growth factor”, or EGF, is a polypeptide which (1) shares substantial sequence similarity with a native EGF; and (2) possesses a biological activity of the native EGF. The native EGF is preferably a mammalian EGF. For example, the native human EGF is a 53-amino acid polypeptide synthesized mainly in the salivary glands and duodenum of normal humans (Carpenter et al., 1979; U.S. Pat. No. 6,191,106). A polypeptide which shares “substantial sequence similarity” with a native EGF is at least about 30% identical with the native EGF at the amino acid level. The EGF is preferably at least about 40%, more preferably at least about 60%, yet more preferably at least about 70%, and most preferably at least about 80% identical with the native EGF at the amino acid level. The phrase “percent identity” or “% identity” with a native EGF refers to the percentage of amino acid sequence in the native EGF which are also found in the EGF analog when the two sequences are aligned. Percent identity can be determined by any methods or algorithms established in the art, such as LALIGN or BLAST.

A polypeptide possesses a “biological activity of EGF” if it is capable of binding to the EGF receptor or being recognized by a polyclonal antibody raised against the native EGF. Preferably, the polypeptide is capable of specifically binding to the EGF receptor in a receptor binding assay.

Thus, the term “EGF” encompasses EGF analogs which are the deletional, insertional, or substitutional mutants of a native EGF. Particularly included as an EGF is the native EGF of any species, transforming growth factor (TGF), or a recombinant modified EGF. Specific example include, but are not limited to, the recombinant modified EGF having a deletion of the two C-terminal amino acids and a neutral amino acid substitution at position 51 (particularly EGF51 gln51; U.S. Patent Application Publication No. 20020098178A1), the EGF mutein (EGF-X₁₆) in which the His residue at position 16 is replaced with a neutral or acidic amino acid (U.S. Pat. No. 6,191,106), the 52-amino acid deletion mutant of EGF which lacks the amino terminal residue of the native EGF (EGF-D), the EGF deletion mutant in which the N-terminal residue as well as the two C-terminal residues (Arg-Leu) are deleted (EGF-B), the EGF-D in which the Met residue at position 21 is oxidized (EGF-C), the EGF-B in which the Met residue at position 21 is oxidized (EGF-A), heparin-binding EGF-like growth factor (HB-EGF), or a fusion protein comprising any of the above. The EGF may also contain additional amino acids added to the native EGF or EGF muteins. For example, EGF-flag derivatives have an 8 amino acid “flag” sequence at the N-terminus, which permits rapid purification of peptides by affinity chromatography using columns containing anti-flag monoclonal antibodies (International Biotechnology Inc.). Other useful EGF analogs or variants are described in U.S. Patent Application Publication No. 20020098178A1, and U.S. Pat. Nos. 6,191,106 and 5,547,935.

The “gastrointestinal system” means the part of the digestive system from stomach to large intestine, including the entire small and large intestines.

A “subclinical infection” is an infection by any pathogen without clinical manifestations.

The term “animal” as used herein is meant to include all members of the animal kingdom such as fish and mammals (including farm animals and even humans).

Methods

EGF can be used to inhibit or treat pathogenic infections of the gastrointestinal tract (U.S. Pat. No. 5,753,622). As shown in Example 1, rabbits pre-treated with EGF did not develop diarrhea even though they were given an E. coli which caused diarrhea in rabbits not treated with EGF. The group which was pre-treated with EGF also excreted the E. coli one day earlier than the untreated group, and E. coli colonization in the gut of the treated animals was significantly reduced by EGF. Therefore, EGF prevented bacterial colonization of the gut, which resulted in early clearance of the bacteria, thereby protecting the animals from bacterial infection and diarrhea.

Similar protective effects were observed in a gastric ulcer model. As shown in Example 2, ulcers were induced in rats, and EGF was given to a group of the ulcer-bearing rats seven days later. The control rats received the same volume of sterile water instead of EGF. For comparison, a third group received a combination of two broad-spectrum antibiotics, streptomycin and penicillin. As expected, the antibiotics-treated rats had significantly less bacterial colonization at the ulcer sites, and the ulcer healed faster than the control rats. The extent of bacterial colonization in the EGF-treated rats was also low and comparable to the antibiotics-treated rats, indicating that EGF effectively inhibited bacterial colonization. Consistent with this result, the ulcers in the EGF treated group also healed faster than those in the control rats which received sterile water only. Therefore, EGF exerted anti-infection and wound healing activities to gastrointestinal epithelia.

The anti-infection effects of EGF are not mediated by direct bacterial growth inhibition. As shown in Example 3, the bacteria incubated in the presence of EGF displayed a growth rate comparable to that of the bacteria without EGF. Therefore, EGF does not inhibit bacterial colonization and infection by directly inhibiting bacterial growth, indicating that EGF most likely interferes with binding of bacteria to, and the subsequent colonization at, the epithelial cells in the gastrointestinal tract.

In normal humans, large amounts of EGF may be found throughout the lumen of the gastrointestinal tract (Konturek et al., 1990). Chronic administration of EGF produces a significant increase in gastrointestinal mucosal DNA, RNA, and protein content, and this proliferative action of EGF is believed to contribute to the normal maintenance of mucosal integrity within the gastrointestinal tract. Other effects of EGF on the gastrointestinal tract are also well-documented. For example, EGF promotes the proliferation and differentiation of intestinal cells during early life, the functional maturation of the pre-weaning intestine, and epithelial proliferation in the adult gut (O'Loughlin et al., 1985; Goodlad et al., 1991; Walker-Smith et al., 1985). EGF has also been shown to upregulate small intestinal absorption of electrolytes and nutrients (O'Loughlin et al., 1994). These results indicate that EGF primarily exerts its functions in the gastrointestinal tract and support the notion that EGF can specifically inhibit adhesion of pathogens to the gastrointestinal epithelia.

Surprisingly, we discovered that EGF is also capable of inhibiting pathogenic infection outside of the gastrointestinal tract. As shown in Examples 4 and 5, we tested the effects of EGF using a variety of other mucosal systems. The results indicate that EGF is capable of inhibiting pathogenic colonization in bladder tissues and kidney epithelial cells. Accordingly, EGF inhibits pathogenic colonization and infection in tissues beyond previous expectation, and thus it can be used to prevent or treat pathogenic infections in a wide variety of infectious conditions. Furthermore, Example 6 also shows that EGF is effective against the infection of widely different pathogens as well.

Accordingly, the present invention provides a method of inhibiting or treating a pathogenic infection in an animal, comprising administering an effective amount of epidermal growth factor to the animal. Preferably, the infection occurs in the urogenital tract, including the kidney, ureter, bladder, urethra, prostate, testes, ovary, fallopian tube, uterus, cervix and vagina.

The present invention is particularly useful for the prevention or treatment of a disease or medical condition in which the etiological factor is pathogenic infections, but it is hard to identify the causative pathogen or to detect symptoms of infection. For example, prostatitis is a common urologic condition which is sometimes difficult to treat effectively. It has been estimated that up to half of all men suffer from symptoms of prostatitis at some time during their lives (Domingue et al., 1998).

There are three kinds of prostatitis: acute bacterial prostatitis, chronic bacterial prostatitis, and chronic idopathic prostatitis. Culture diagnosis of acute bacterial prostatitis is straightforward, while chronic bacterial prostatitis is a more subtle illness, characterized by relapsing, recurrent urinary tract infection, and persistence of bacteria in the prostatic secretory system despite multiple courses of antibacterial therapy. Chronic idiopathic prostatitis, on the other hand, may or may not involve excessive number of inflammatory cells in prostatic secretions or culturally documented bacteriuria. In fact, the prostatic secretions from many patients appear normal. Recently, it has been suggested that chronic idiopathic prostatitis is associated with pathogenic infections. Various bacteria, and to a lesser extent mycobacteria, fungi, parasites and viruses have been associated with this disease (Domingue et al., 1998). Since EGF is capable of inhibiting infections of a wide variety of pathogens, it is ideal to use EGF to treat prostatitis, even if the causative agent can not be identified.

Cystitis is a condition of inflammation in the bladder generally classified into two types, bacterial cystitis and interstitial cystitis. Bacterial cystitis is resulted from bacterial infection, and therefore EGF is an ideal therapeutic agent in the treatment of bacterial cystitis. Interstitial cystitis is a poorly-understood medical condition for which the present invention may also be particularly useful. Interstitial cystitis is a type of bladder condition found predominantly in women. Generally agreed criteria for its diagnosis are the frequency, urgency, and pain of urination; a low-capacity hypersensitive bladder; and mucosal haemorrhages as well as tearing on bladder distention. However, there are no specific histopathological changes that are diagnostic of interstitial cystitis. Despite being described over 80 years ago, it remains a disease of undetermined etiology and poor treatment outcomes. It has been suggested that infection is an etiological factor, perhaps by playing a role in the initial stage of this condition, although studies using light microscopy, electron microscopy, serology and molecular biological techniques have not consistently isolated any microorganism (Rosamilia et al., 2000). Therefore, EGF can be used to treat interstitial cystitis, particularly to prevent further progress of the disease beyond the initial stage.

EGF can also be used to prevent or treat pathogenic infections which occur subsequent to the infliction of another medical condition, for example, a wound. The wounds contemplated in the present invention are typically wounds in the skin or mucosal surfaces, and include, for example, burns, cuts, punctures, ulcers and tears. However, EGF may be useful to any wound which is susceptible to pathogenic infections. To prevent pathogenic infections, it is preferable that EGF is administered to the subject bearing a wound before there is any indication of pathogenic infections. EGF may be administered according to any method or route established in the art. Preferably, EGF is administered orally or topically at/near the wound. If pathogenic infections have occurred, EGF can still be administered to ameliorate and treat the infections.

It is contemplated that in addition to the native EGF, any EGF analog which has the activity of inhibiting pathogenic colonization is useful in the present invention. The ability to inhibit pathogenic colonization of any EGF analog, which possesses substantial sequence identity and biological activity with the native EGF, may be determined according to the methods disclosed herein.

The EGF should be administered in a formulation and through a route which are consistent with its purpose. For example, for urogenital infections, the EGF is preferably administered topically, including luminal and intracavital administrations. For instance, the EGF may be administered in the form of a douche, solution, emulsion, cream, ointment, gel, paste, suppository or catheter delivery. The EGF may also be delivered by any way that results in appearance of the EGF in the target tissue. For example, the EGF may be administered systemically, or delivered using a vehicle that leads to release of the EGF. Such vehicle includes, but is not limited to, an expression vector encoding the EGF, a genetically modified bacterium, yeast or particularly virus expressing the EGF, or a genetically modified plant or parts thereof expressing the EGF.

The following examples are offered to illustrate this invention and are not to be construed in any way as limiting the scope of the present invention.

### EXAMPLES

In the examples below, the following abbreviations have the following meanings. Abbreviations not defined have their generally accepted meanings.
° C.=degree Celsius
hr or h=hour
min minute
μM=micromolar
mM=millimolar
M=molar
ml=milliliter
μl=microliter
mg=milligram
μg=microgram
rpm=revolutions per minute
FBS=fetal bovine serum
FCS=fetal calf serum
DTT=dithiothrietol
DMEM=Dulbecco's modified Eagle's medium
CFU=colony forming unit
PBS=phosphate buffered saline
EGF=epidermal growth factor
PDGF=platelet derived growth factor

### Example 1

### Effects of EGF on Intestinal Infection

A preliminary study using 15 New Zealand white rabbits (6 week old, 500-700 g) was carried out to test the hypothesis that EGF may protect the animals from intestinal colonization by E. coli. Animals were divided in three groups: 1) unmanipulated controls, 2) animals orally infected with E. coli, and 3) animals orally infected with E. coli and given daily oral dosages of 60 μg recombinant human EGF (Austral Biologicals, San Ramon, Calif. 94583) for 10 days starting 3 days prior to infection. All animals were checked daily for weight gain, food intake, rectal passage of E. coli, and presence of diarrhea. The results are summarized in Table 1.

TABLE 1

| | Cumulative | Feed | Mucosal | Wet Weight³ | |
|---|---|---|---|---|---|
| | Weight Gain¹ | Efficiency² | ileum | prox. colon | E. Coli⁴ |
| CONTROL | 358 ± 15 | 2.2 ± 0.2 | 122 ± 5 | 198 ± 15 | — |
| INFECTED | 293 ± 33 | 3.3 ± 1.3 | 116 ± 6 | 170 ± 11 | 4.41 ± 0.23 |
| INFECTED + | 335 ± 24 | 1.8 ± 0.3 | 128 ± 6 | 195 ± 17 | 3.99 ± 0.24 |
| EGF | | | | | [62%] |

Values are means ± Standard error from mean of 5 animals per group 7 days after inoculation, [%] percent bacterial clearance
¹grams
²Food intake/weight gain
³milligram/cm
⁴Log 10 CFU (per cm proximal colon)

Clinically, in untreated infected animals, rectal swabs were positive for E. coli 2 days after inoculation and 3 out of 5 rabbits showed signs of diarrhea by day 7. In contrast, infected animals given daily doses of 60 μg EGF excreted E. coli a day earlier and did not show signs of diarrhea. Controls had no diarrhea or E. coli (either from rectal swabs or in the intestines at necropsy). Compared to controls, 7 days after infection, infected animals had a reduced cumulative weight gain, poorer feed conversion efficiency, and decreased mucosal wet weights in the ileum and proximal colon. EGF treatment reduced bacterial colonization in the proximal colon by 62%, protected mucosal weight in ileum and colon, and improved feed conversion efficiency and weight gain (Table 1). Feed efficiency and weight gain in treated-infected animals were comparable to noninfected controls.

### Example 2

### The Effects of EGF on Bacterial Colonization of Gastric Ulcers

Gastric ulcer induction results in markedly elevated levels of bacterial colonization at the ulcer site, which delays ulcer healing (Elliott et al., 1998). In order to examine the effects of EGF on preexisting bacterial colonization at ulcer sites, ulcers were induced using a rat ulcer model as follows.

Male Wistar rats weighing 175-200 g were obtained from Charles River Laboratories (St. Constant, PQ, Canada). The animals had free access to standard pellet chow and tap water throughout the experiment, except that food was made unavailable during a fasting period of 18-24 hours prior to ulcer induction. Ulcers were induced using a method modified from the model previously described (Okabe and Pfeiffer, 1972). Briefly, under halothane anesthesia, a midline laparotomy was performed and the stomach was gently exteriorized. The barrel of a 3-ml syringe, which had been cut and filed smooth, was placed on the serosal surface of the stomach in the corpus region. Half a milliliter of 80% acetic acid (vol/vol) was instilled into the barrel of the syringe and allowed to remain in contact with the stomach for 1 min, after which time it was aspirated off and the area was gently rinsed with sterile saline. The area exposed to acetic acid was 59.7 mm². Gastric ulcer area was determined as follows. The rats were killed by cervical dislocation, and the stomach was removed and pinned out on a wax block. A paper grid with an area of 25 mm² was placed alongside the ulcer, which was then photographed. Ulcer area was determined by planimetry, using 5× enlargements of the photographs. The area of ulceration in pixels was then converted to units of square millimeters, using the paper grid as a reference. All planimetric determinations were performed using coded photographs such that the observer was unaware of the treatment the rats had received.

On the seventh day after ulcer induction, a 7-day treatment period was initiated during which EGF (1 or 100 μg/kg) was orally administered once daily. The vehicle for EGF was sterile water, and control rats received the same volume of sterile water instead of EGF. For comparison, a third group of rats received twice-daily oral treatment of a combination of streptomycin (336 mg/ml; 0.25 ml) and penicillin (168 mg/ml; 0.25 ml), which are broad-spectrum antibiotics known to inhibit bacterial infections. At the end of the 7-day treatment period, the rats were killed by cervical dislocation, the stomach was removed for ulcer area determination, and tissue samples were taken for bacterial culturing. The bacterial levels recovered from the EGF-treated or antibiotics-treated rats were calculated and expressed as percentages of the average number of bacteria recovered from the control group (vehicle alone).

The results are as follows. Rats receiving vehicle over the seven-day treatment period had a mean bacterial level of 6.5 log CFU/g tissue at the ulcer site, a level significantly (p<0.01) higher than those obtained from tissue cultures taken from the stomach of rats without ulcers (3-4 log CFU/g tissue, see Elliott et al., 1998). Administration of EGF at either 1 or 100 μg/kg significantly (p<0.01) reduced bacterial levels (5.0±0.4 and 5.3±0.3 log CFU/g tissue, respectively) relative to the rats receiving vehicle alone. Treatment with the streptomycin/penicillin combination also resulted in a marked reduction in bacterial colonization at the ulcer sites (4.9±0.3 log CFU/g tissue). Therefore, EGF was comparable to antibiotics in its effect against bacterial colonization at gastric ulcer sites.

### Example 3

### EGF does not Directly Inhibit Bacterial Growth

The effects of EGF on bacterial growth were determined in vitro. Three bacterial isolates were used for these studies: 1) gram-positive Enterococcus faecalis isolated from fresh rat feces as a single colony grown on a TSB agar plate for 18 h at 37° C., 2) gram-negative Escherichia coli isolated from fresh rat feces as a single colony grown on a TSB agar plate for 18 h at 37° C., and 3) a streptomycin-resistant strain of E. coli (C-25) that has previously been shown to delay healing of gastric ulcers in rats (Elliott et al., 1998). The E. faecalis and E. coli isolated from fresh feces were identified as such by the Veterinary Pathology Laboratory (Alberta, Edmonton, AB, Canada) using standard bacterial identification sensitivity assays. All bacterial stock cultures were stored at −70° C. in TSB (Difco Laboratories, Detroit, Mich.) coated onto Microbank porous beads (Pro-Labs Diagnostics, Richmond Hill, ON, Canada). In a series of three experiments, log phase bacteria (10³ CFU/ml) were added in duplicate to wells on a 96-well plate containing TSB with either no EGF (control) or 10 μM EGF, in a total volume of 100 μl/well. This concentration was chosen to reflect the higher end of EGF levels that may be encountered by gastrointestinal bacteria in vivo (Gregory, 1985) and is consistent with previous studies using similar experimental protocols of oral EGF administration in infected animals (Buret, et al., 1998). At 1-h intervals (0-5 h postinoculation), viable bacterial cells in each well were counted by serial dilution and culture on TSB agar plates (for cocci) or MacConkey agar plates (for rods and E. coli) for 18 h at 37° C. Bacterial numbers are expressed as loglo CFU per milliliter.

### Example 4

### EGF Inhibits Colonization of E. Coli in Bladder Tissue

Bladder tissue samples of 1 cm² were excised from a New Zealand White rabbit and placed in a 24 well plate. Half of the wells then received human recombinant EGF (Austral Biologicals, 10 μM final concentration) and the other half received the vehicle, sterile PBS, to serve as controls. Fifteen minutes later, 2×10⁸ E. coli (human urinary tract infection isolate K1:08AC:H7) were added to each well and co-incubated in 900 μl DMEM tissue culture medium for 3 hours at 30° C. and 5% CO₂. The tissue samples were then washed in sterile PBS, weighed and homogenized. E. coli colonization was assessed by serial dilution and spot-plating into McConkey agar plates followed by incubation overnight.

The results show that EGF treatment reduces bladder colonization by E. coli (Table 2).

TABLE 2
Colonization of E. coli on bladder tissue
samples in the presence and absence of EGF

| | Colonization | |
|---|---|---|
| | (Log CFU/g tissue) | Inhibition of colonization |
| | mean ± SEM | (% reduction vs. control) |
| Control (n = 6) | 7.9 ± 0.1 | |
| 10 μM EGF (n = 6) | 7.6 ± 0.1* | 47.5% |

*p < 0.05 vs. control.

These results indicate that EGF significantly inhibits bacterial colonization in a bladder infection.

### Example 5

### Effects of EGF on Other Cells of Epithelial Origins

The effects of EGF on colonization of the protozoan parasite Cryptosporidium parvum on bovine kidney epithelial cells (MDBK and NBL-1) or human intestinal epithelial cells (CaCo2 and SCBN) were investigated. The cells were given 1 μM EGF, or vehicle alone to serve as controls. Fifteen minutes later, the parasite was added to the cells, and the extent of colonization (% of cells infected by parasites) was determined after 24 hours.

The results indicate that administration of EGF significantly reduced Cryptosporidium parvum colonization in all cell lines. Therefore, EGF has anti-infective activities in intestinal epithelial cells as well as cells from other mucosal systems, in this case kidney epithelial cells. EGF is also effective in inhibiting the infections of pathogens other than bacteria, in this case the parasite Cryptosporidium parvum. Furthermore, EGF is effective across species and inhibits pathogenic infections in human and bovine cells.

### Example 6

### Effect of EGF on Other Bacteria and Parasites

This experiment was conducted in order to assess the effects of EGF on the colonization of other pathogens, such as Salmonella typhimurium and E. coli K-12, on human epithelial cells.

A. Two×10⁸ human pathogenic Salmonella typhimurium or E. coli were added to the apical surface of confluent human CaCo2 monolayers grown on Transwell membranes (porosity 3.0 μm). Monolayers received apical EGF (100 μm or 10 μm) or PBS 15 min prior to infection. Each hour post infection (0-7 h), medium under the membrane was replaced and bacterial transepithelial migration rate (CFU/h) was calculated.

The results show that 100 μm EGF delayed the initial E. coli translocation by 1 hour and inhibited the rate of invasion by more than 95% thereafter. Translocation of Salmonella typhimurium was completely abolished in monolayers treated with 100 μm EGF, and was inhibited by more than 90% by 10 μm EGF.

B. To further investigate the effects of EGF on parasites, the following experiment was conducted to determine the effects of EGF on the infection of a parasite in gerbils.

Gerbils were infected with 200,000 trophozoites (Giardia lamblia, S2 isolate). One group received daily oral PBS, and the results were compared to data obtained from animals given daily oral EGF (100 μm/kg) starting 3 days prior to infection. Jejunal samples were obtained 6 days post-infection and trophozoites colonizing the intestinal mucosa were counted.

The results are summarized in table 3 below, which demonstrate that EGF treatment significantly inhibits intestinal colonization by Giardia lamblia.

TABLE 3
Numbers of trophoziotes recovered from the jejunum
of gerbils infected for 6 days with Giardia lamblia

| | PBS | EGF |
|---|---|---|
| Trophoziote numbers: 10⁴/cm | 17.9 ± 2.9 | 10.7 ± 1.0* |
| jejunum ± standard error | | |

*p < 0.05.

We then used the human small intestinal epithelial cell line SCBN to study host-cell parasite interactions in giardiasis and effect of EGF. We found that G. lambilia disrupted tight-junctional ZO-1 of SCBN cells and significantly increased paracellular permeability. Pre-treatment with EGF, however, prevented these abnormalities and inhibited attachment of live trophozoites to the cells.

C. The effect of EGF on Helicobacter infection was also assessed. Female C57BL/6 mice aged 6-8 weeks were housed in autoclaved cages and given unlimited access to sterile food and water. Animals were randomly assigned to one of the following groups: 1) uninfected control, 2) infected-untreated (vehicle), and 3) infected-EGF treated. Animals were infected orogastrically with a 0.2 ml inoculum containing 1×10⁹ live Helicobacter pylori (SS 1 strain) suspended in sterile phosphate-buffered saline (PBS) on days 0, 2 and 4. Uninfected animals received sterile PBS alone. Infection was allowed to progress for 2 and 10 weeks.

Treatment was orally administered daily for 10 days prior to sacrifice. EGF treated animals received mouse recombinant EGF (100 μg/kg in sterile PBS) and sham-treated animals received sterile PBS. At sacrifice tissue was collected from the stomach for assessment of H. pylori colonization as follows.

Tissue samples were diluted 1:10 (w:v) in sterile PBS, homogenized and serially diluted on selective Columbia Blood Agar plates (containing 7% heat-inactivated horse serum, 10 mg/L vancomycin, 5 mg/L trimethoprim, 20 mg/L bacitracin, 10 mg/L nalidixic acid, 2500 IU/L polymyxin B). Plates were incubated at 37° C. in a microaerophilic chamber and after 5 days assessed for colony forming units.

The results show that EGF dramatically reduced the numbers of H. pylori that were isolated from infected animals. Thus, the infected-treated group produced orders of magnitude less H. pylori than the infected-untreated counterparts. As expected, the uninfected control resulted in no bacteria. Therefore, EGF is highly effective against pathogenic infection.

### Example 7

### Effect of EGF on Weight Gain

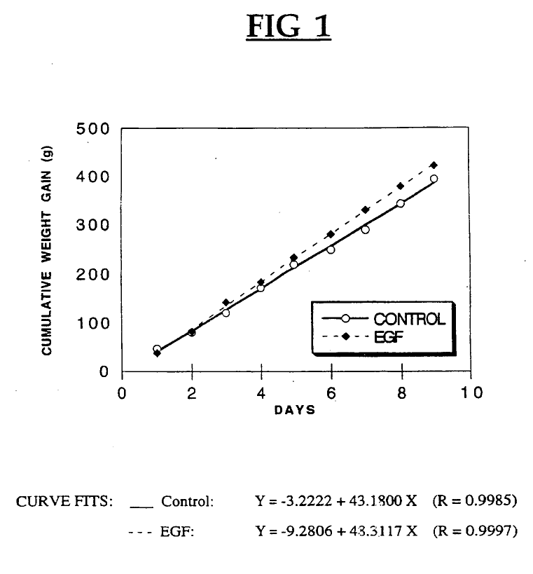

EGF was tested for its potential benefits on weight gain. One group of New Zealand white rabbits (6 week old, 500-700 g) received daily oral doses of recombinant human EGF (100 μg/kg body weight) and control animals were given saline only. At 9 days, EGF-treated animals had a mean cumulative weight gain of 422±27 g (n=10) while controls only gained 394±16 g (n=11). Referring to FIG. 1, the slope of the linear regression curve of weight gain in EGF treated animals was significantly greater (P 0.002) than that of untreated controls. Given the linear aspect of both curves, continued feeding with EGF is likely to produce a steadily increasing effect on weight gain.

These results indicate that EGF as a food additive and as a gastrointestinal therapeutic agent may promote the acceleration of weight gain in healthy animals.

### Example 8

### Inhibition of EGF to Decrease Weight Gain

Based on the hypothesis that down regulation of specific components of the EGF signalling cascade may inhibit nutrient absorption, the effects of tyrophostin 51 (Sigma), (a tyrosine-kinase inhibitor), on nutrient absorption and brush border ultrastructure were tested. Tyrophostin 51 is a specific inhibitor of tyrosine kinase, which is a critical element of the EGF signalling cascade. Two experimental 10 cm jejunal loops separated by a 1 cm blind loop were tied off in New Zealand White rabbits (8 week old, 700-1000 g). Tyrophostin (10 μM in 1.5 mL of saline) was injected into one experimental loop. The other loop received vehicle alone as a control. After one hour, brush border membrane vesicles were prepared from both loops and assessed for nutrient (D-glucose) absorption. In addition, using transmission electron microscopy, the height of brush border microvilli was measured. The height of the brush border microvilli is a parameter that has been established as the limiting factor for overall brush border surface area in the inventor's previous studies. The preliminary findings demonstrate that treatment with tyrophostin 51 decreases nutrient uptake (26.9±2.8 nmol/min/mg Prot) and microvillus height (1.05±0.20 μm) when compared to control values (transport=36.6±1.8 nmol/min/mg Prot; microvillus height=1.90±0.15 μm). The inventor's have previously demonstrated that levels of intestinal nutrient absorption and diffuse microvillus membrane surface area correlate with weight gain in a number of different models. Thus, it is predicted that treatment with tyrophostin may promote decreased weight gain or weight loss and therefore may be useful in the treatment of obesity. As well, such treatment may be used to reduce the intestinal uptake of toxic or adverse substances.

The above describes new utilities for EGF. In particular, EGF has been shown to prevent gastrointestinal colonization by pathogens and to promote weight gain in animals. Consequently, EGF is a very useful agent that can be used to increase production in the animal industry such as the beef, pig and poultry industry. In addition, EGF treatment may have clinical benefits in humans (i.e. during Crohn's disease, gastrointestinal infection, traveller's diarrhea, etc.).

Inhibitors of EGF may decrease nutrient absorption in the intestine and as such may be useful in treating obesity or in preventing absorption of toxins.

One skilled in the art will appreciate that the present invention relates to new utilities of EGF and inhibitors of EGF. The examples described are meant to be models to exemplify the invention and not to limit the invention. The mode of administration, the formulation and the dose of the EGF or EGF inhibitor can be varied depending on the particular utility. For example, when treating young farm animals the EGF can be administered orally in the feed or drinking water of the animal. The dose range can be varied from 10-10,000 μg/kg per day.

We claim:

1. A method of promoting of weight gain in an animal comprising administering an effective amount of an EGF to the animal.

2. The method of claim 1, wherein the EGF is administered in the feed of the animal.

3. The method of claim 1, wherein the EGF is administered in the drinking water of the animal.

4. The method of claim 1, wherein the EGF is administered orally.

5. The method of claim 1, wherein 10 to 10,000 μg/kg of the EGF is administered per day to the animal.

6. The method of claim 5, wherein 10 to 100 μg/kg of the EGF is administered per day to the animal.

7. The method of claim 1, wherein the EGF is administered for at least nine days.

8. The method of claim 1, wherein the animal is an adult animal.

9. The method of claim 1, wherein the animal is not a newborn animal.

10. The method of claim 1, wherein the animal is a young animal.

11. The method of claim 1, wherein the animal is a juvenile animal.

12. The method of claim 1, wherein the animal is a healthy animal.

13. The method of claim 1, wherein the animal has an infection.

14. The method of claim 1, wherein the animal is a farm animal.

15. The use of claim 14, wherein the animal is a food producing animal.

16. The method of claim 1, wherein the animal is a human.

17. The method of claim 1, wherein the EGF is a recombinant EGF.

18. The method of claim 1, wherein the EGF is selected from the group consisting of a transforming growth factor (TGF), a recombinant modified EGF having a deletion of the two C-terminal amino acids and a neutral amino acid substitution at position 51, EGF-X₁₆, EGF-D, EGF-B, EGF-C, EGF-A, HB-EGF, and a fusion protein comprising any of the above

19. The method of claim 18, wherein the EGF is selected from the group consisting of a native EGF, EGF51 gln51, EGF-D, EGF-X₁₆, TGF and HB-EGF.

20. A method of treating obesity, comprising administering to an animal an effective amount of an inhibitor of EGF activity.

21. The method of claim 20, wherein the inhibitor is a EGF-receptor tyrosine kinase inhibitor.

22. A method of preventing absorption of an adverse substance comprising administering to an animal an inhibitor of EGF activity.

23. The method of claim 22, wherein the inhibitor is an EGF-receptor tyrosine kinase inhibitor.

24. The method of claim 22, wherein the adverse substance is a toxin.

25. A method of inhibiting or treating a pathogenic infection of a mucosal surface of an animal, comprising administering an effective amount of an epidermal growth factor (EGF) to the animal.

26. The method of claim 25, wherein the infection is selected from the group consisting of bacterial infections, yeast infections, parasitic infections and viral infections.

27. The method of claim 25, wherein the EGF is administered topically.

28. The method of claim 25, wherein the EGF is a recombinant EGF.

29. The method of claim 25, wherein the EGF is selected from the group consisting of a transforming growth factor (TGF), a recombinant modified EGF having a deletion of the two C-terminal amino acids and a neutral amino acid substitution at position 51, EGF-X₁₆, EGF-D, EGF-B, EGF-C, EGF-A, HB-EGF, and a fusion protein comprising any of the above

30. The method of claim 29, wherein the EGF is selected from the group consisting of a native EGF, EGF51 gln51, EGF-D, EGF-X₁₆, TGF and HB-EGF.

31. The method of claim 25, wherein the mucosal surface is located in the digestive tract, respiratory tract, urogenital tract, ocular surface, mammary gland or prostate of the animal.
